(19) United States
(12) Patent Application Publication
(10) Pub. No.: US 2003/0040047 A1
(43) Pub. Date: Feb. 27, 2003

(54) Method for producing a recombinant protein

(75) Inventors: Mike Farwick, Bielefeld (DE); Markus London, Dormagen (DE); Juergen Dohmen, Meerbusch (DE); Ulrike Dahlems, Krefeld (DE); Gerd Gellissen, WUelfrath (DE); Alexander W. Strasser, Duesseldorf (DE)

Correspondence Address: MORGAN, LEWIS & BOCKIUS LLP, 1701 MARKET STREET, PHILADELPHIA, PA, 19103-2921, US

(73) Assignee: Rhein Biotech

(21) Appl. No.: 09/999,392

(22) Filed: Oct. 31, 2001

Related U.S. Application Data
(63) Continuation of application No. PCT/EP00/04062, filed on May 5, 2000.

(30) Foreign Application Priority Data
May 5, 1999 (DE) EP 199 20 712.7

(51) Int. Cl. C12P 21/02; C12N 1/18; C12N 15/74

(52) U.S. Cl. 435/69.1; 435/254.21; 435/483

(57) ABSTRACT

The present invention relates to methods for producing recombinant proteins, in particular recombinant secretory proteins, to a method for identifying nucleic acid molecules the expression products of which permit improved secretion of a recombinant secretory protein, to the use of molecules thus identified for enhancing secretion of heterologous proteins, and to corresponding kit systems.

The method according to the invention makes provision for expression in a suitable host cell of a nucleic acid coding for a secretory protein together with a nucleic acid coding for a polypeptide having the biological activity of the eukaryotic translation initiation factor 4 E and/or for a polypeptide having the biological activity of a CaM kinase. It was found that coexpression of the translation initiation factor gene and/or of the CaM kinase gene leads to improved secretion of a desired recombinant protein from the cell.

The present invention relates to methods for producing recombinant proteins, in particular recombinant secretory proteins, to a method for identifying nucleic acid molecules the expression products of which permit improved secretion of a recombinant secretory protein, to the use of molecules thus identified for enhancing the secretion of heterologous proteins, and to corresponding kit systems.

Many active compounds contained in medicaments are now no longer manufactured by chemical synthesis or by extraction from natural plant or animal sources and purification, but with the aid of recombinant DNA technology. One benefit of this technology is that the proteins can be obtained in practically unlimited quantities in well-characterized hosts.

Recombinant proteins may be obtained more effectively if they are secreted, in order to enable them then to be obtained from the cell supernatant. This considerably simplifies processing, as the protein is already present in a relatively pure form, and the number of complex purification steps can be reduced.

Yeasts and filamentous fungi are organisms frequently employed for the production of secretory proteins in large quantities. Their advantage is that they can be maintained relatively easily in cell culture, and return high yields of secreted proteins. In addition, they are capable of posttranslational modification of the produced proteins, e.g. by glycosylation.

For attainment of expression of recombinant DNA sequences, nucleic acid sequences encoding a protein of interest are generally cloned into a vector containing, as its functional elements, a promoter and polyadenylation signals, and also termination elements. The vector is inserted into a host cell by suitable methods, such as electroporation, calcium- or liposome-mediated transfection, etc. A DNA sequence of this kind may also contain a secretion leader sequence at its 5′ end, which causes the recombinant translation product to be directed into the secretory apparatus of the host cell.

The insertion of foreign DNA into an expression system of this kind is, however, no guarantee of effective gene expression. Inefficient transcription and/or translation, rapid degradation of the messenger RNA (mRNA), and instability of the protein product are frequent problems. In addition, the recombinant protein may be processed and modified incorrectly and/or unsatisfactorily by the host cell. Recombinant proteins are often secreted very inefficiently in baker's yeast, Saccharomyces cerevisiae, for example. This expression system is therefore unsatisfactory for many applications. Under certain circumstances, however, production may be improved in such cases by the simultaneous production of further proteins which permit efficient transcription and/or translation, processing, stabilization, modification, and distribution to certain cell compartments. A series of S. cerevisiae mutants has been produced by this means which proved capable of eliminating potential bottlenecks in the processes extending from translation to secretion of a recombinant protein. Mutations of this kind have been described by Smith et al. for example for the genes ssc1 and ssc2 from S. cerevisiae and by Sakai et al. 1988, Hinnen et al., 1994 and Gellissen and Hollenberg, 1997 for the genes rgr1 and ose1. An alternative approach exploits the overexpression of heterologous genes introduced from other organisms, such as the overproduction of protein disulphide isomerase (PDI) or of the chaperone BiP, or the use of components of the mammalian signal peptidase complex in baker's yeast (Shusta et al. 1998; WO 98/13473).

Methylotrophic yeasts, i.e. yeasts capable of exploiting methanol as their sole source of energy and carbon, are already known to hyperglycosylate recombinant proteins less strongly than S. cerevisiae. In addition, methylotrophic yeasts have the advantage of secreting heterologous proteins relatively efficiently. The same applies, for example, to Kluyveromyces lactis, Aspergillus niger and Schizosaccharomyces pombe (Giga-Hama and Kumagai, 1997; Gellissen and Hollenberg, 1997; Hollenberg and Gellissen, 1997). Even where methylotrophic yeasts are employed as the expression hosts, however, only a limited number of recombinant proteins can be produced and secreted. There is therefore a substantial need for a system which enables any secretory protein of interest to be secreted efficiently. Furthermore, methods are required which enable recombinant proteins to be produced with substantially correct secondary modifications, i.e. the modifications observed following production of the protein in its natural host cell.

The object of the present invention is therefore to provide a means for efficient secretion of recombinant proteins and where applicable for the production and/or secretion of recombinant proteins with secondary modifications which are largely similar to the secondary modifications produced in the natural host.

According to the invention, this object is achieved by a method for producing a recombinant secretory protein, said method comprising the following steps:

a) expressing a nucleic acid coding for the secretory protein in a suitable host cell, together with a nucleic acid coding for a polypeptide having the biological activity of the eukaryotic translation initiation factor 4E (elF4E) and/or for a polypeptide having the biological activity of a Ca²⁺/calmodulin-dependent protein kinase (CaM kinase), the nucleic acid coding for a polypeptide having the biological activity of elF4E or CaM kinase being under the control of a promoter P1;

b) secreting the protein from the cell, and

c) obtaining the secreted protein.

The eukaryotic initiation factor 4E (elF4E) has an important function in initiation of translation. It forms part of the “cap binding” complex (elF4F) and is responsible for binding this complex and the 5′-cap structure of the mRNA (Sonenberg, 1996). Eukaryotic Type II Ca²⁺/calmodulin-dependent protein kinases (CaM kinases) phosphorylate a range of different target proteins and are therefore involved in regulation of the energy metabolism, the cell cycle, and the ion permeability, etc. (Schulman, 1993).

The term “biological activity” of the eukaryotic translation initiation factor 4E and of the Ca²⁺/calmodulin-dependent protein kinase refers herein to the activity of these enzymes as described in the literature. The biological activity of elF4E is determined herein as described by Altmann & Trachsel (1989), that of CaM kinase as described by Ohya et al. (1991).

The promoter P1 may be any desired promoter which functions as a promoter in the selected host cell. Numerous examples of such promoters can be found in the state of the art (such as Sambrook et al., 1989).

The method enables the protein of interest to be produced and secreted efficiently by coexpression of the genes for the translation initiation factor 4E and/or the Ca²⁺/calmodulin-dependent protein kinase or their derivatives together with expression of a gene for the protein of interest.

Strong overproduction of a recombinant protein has been observed to overload the transcription/translation and secretory machinery of the host cell, resulting in its growth being retarded or even arrested. A frequent bottleneck lies in initiation of translation, upon which the ATP-dependent initiation factor 4E was found by the inventors to have a potentially limiting effect. This bottleneck can be eliminated by coexpression of the gene for this translation initiation factor with a recombinant DNA sequence. Surprisingly, coproduction of the translation initiation factor 4E also influences the secretion efficiency, however. The inventors observed a strong increase in secretion of the desired secretory protein as soon as the latter was coproduced with 4E. In addition, the present invention demonstrates for the first time that coproduction (Pausch et al., 1991; Ohya et al., 1991) of CaM kinase with a recombinant protein also leads to efficient secretion of the recombinant protein concerned. The mechanism of action leading to increased protein secretion remains unexplained in both cases, however.

The method according to the invention thus enables even strongly overproduced proteins to be secreted efficiently, thus allowing extremely high yields to be achieved even for secretory proteins. Production may be increased in this case by a single measure or by several measures in combination. The choice of the vector alone may influence the copy number per cell. 2μ-based vectors are present in between 5 and over 100 copies per cell; 20 to 40 copies are usually present per yeast cell. Integrative vectors may be present in as many as 80 to 150 copies per cell.

The choice of selection system may also be used to influence the copy number of the vector. The use of tryptophane-auxotrophic mutants the auxotrophy of which is complemented by the selection marker TRP1 on the plasmid leads to amplification of the plasmid in the auxotrophic cell. One means by which the copy number can be increased even further is by the use of LEU2 auxotrophic strains and by complementation with the Leu2d allele. The Leu2d allele is an LEU2 gene with a largely deleted promoter and correspondingly weak transcription. The cell's urgent need for LEU2 protein leads to extreme amplification of the plasmid, including amplification of a structural gene for a desired recombinant protein that is located upon it.

An additional factor determining the efficiency of the production system is the strength of the promoter P2 preceding the structural gene for the desired gene to be expressed. Inducible and non-inducible promoters of differing strength are known to persons skilled in the art. Refer in this context to the manual by Sambrook et al., which describes a number of promoters. The GAL1 promoter or the PDC1 promoter are preferably used for production in yeast.

The proteins thus manufactured may be obtained easily from the cell supernatant by removal of cell residue from the latter by centrifugation, and further processed by suitable purification methods, such as ion exchange chromatography, affinity chromatography, gel electrophoresis, etc.

In a preferred embodiment of the above method, the nucleic acid coding for the polypeptide having the biological activity of the translation initiation factor 4E is selected from the following group:

i) a nucleic acid coding for elF4E from Saccharomyces cerevisiae (CDC33);

ii) a nucleic acid coding for a homologue of elF4E having the biological activity of elF4E,

iii) a nucleic acid derived from the nucleic acid set forth in i) or ii) by degeneration of the genetic code, by deletion, insertion, addition and/or nucleotide exchange, said nucleic acid coding for a polypeptide having the biological activity of elF4E;

iv) a fragment of one of the nucleic acids set forth in i) to iii), said fragment coding for a polypeptide having the biological activity of an elF4E;

v) a nucleic acid capable of hybridizing with a sequence complementary to the nucleic acids set forth in i) to iv).

“Homologue” shall refer in this context to proteins exhibiting at least 60% homology with the amino acid sequence of the translational initiation factor 4E from Saccharomyces cerevisiae (Brenner et al., 1988). In preferred embodiments, the homology shall be 70% or 80%, and in particularly preferred embodiments, 90% or 95%. A homology of 98% or 99% is most preferred. The homology may be calculated in this case as described by Pearson and Lipman, 1988.

The term “homology” as employed in the art refers to the degree of affinity between two or more proteins, as determined by comparison of the amino acid sequences by known methods, e.g. computer-aided sequence comparison (basic local alignment search tool, S. F. Altschul et al., J. Mol. Biol. 215 (1990), 403-410)). The percentage of “homology” is derived from the percentage of identical regions in two or more sequences in consideration of gaps or other particular sequence features. In the main, dedicated computer programs are employed in conjunction with algorithms which make allowance for the particular requirements.

Preferred methods for determination of the homology first generate the greatest matches between the sequences being compared. Computer programs for determining the homology between two sequences include, but are not limited to, the GCG program package, including GAP (Devereux, J., et al., Nucleic Acids Research 12 (12): 387 (1984); Genetics Computer Group University of Wisconsin, Madison, (Wis.)); BLASTP, BLASTN and FASTA (Altschul, S. et al., J. Mol. Biol. 215:403-410) (1999)). The BLASTX program can be obtained from the National Centre for Biotechnology Information (NCBI) and from other sources (BLAST Manual, Altschul S., et al., NCB NLM NIH Bethesda, Md 20894; Altschul, S., et al., Mol. Biol. 215:403-410 (1990)). The well-known Smith Waterman algorithm may also be used to determine homology.

Preferred parameters for the amino acid sequence comparison comprise the following:

Algorithm: Needleman and Wunsch, J. Mol. Biol 48:443-453 (1970)

Comparison matrix: BLOSUM 62 described by Henikoff and Henikoff, PNAS USA 89 (1992), 10915-10919

Gap penalty: 12

Gap length penalty: 4

Threshold of similarity: 0

The GAP program is also suitable for use with the above parameters. The above parameters are the default parameters for amino acid sequence comparisons; gaps at the ends do not reduce the homology value. Where very short sequences are compared with the reference sequence, it may also be necessary to increase the expectation value to up to 100,000, and, if necessary, to reduce the word size to as low as 2.

Other exemplary algorithms, gap opening penalties, gap extension penalties, comparison matrices including those set forth in the Program Manual, Wisconsin Package, Version 9, September 1997, may be employed. The selection will depend upon the specific comparison being made, and also upon whether the comparison is between pairs of sequences, in which case GAP or Best Fit are preferred, or between one sequence and a large database of sequences, in which case FASTA or BLAST are preferred.

A 60% match obtained by means of the above algorithm shall be referred to as 60% homology in the context of the present application. Higher degrees of homology shall be treated accordingly.

The object of the invention is further achieved by a method for producing a recombinant protein, comprising the following steps:

a) expressing a nucleic acid coding for the recombinant protein in a suitable host cell, together with a nucleic acid coding for a polypeptide having the biological activity of a Ca²⁺/calmodulin-dependent protein kinase (CaM kinase), said nucleic acid coding for a polypeptide having the biological activity of CaM kinase being under the control of a promoter P1;

b) obtaining the protein.

The protein of interest is produced efficiently by coexpression of the gene for a recombinant protein together with the gene for a Ca²⁺/calmodulin-dependent protein kinase, and can be obtained either from the cell supernatant (in the case of a secretory protein) or from the cell lumen (in the case of a nonsecretory, intracellular protein). In addition to an efficient production, coexpression of the gene for a desired protein with the gene for CaM kinase offers the substantial benefit that the hyperglycosylation of a recombinant protein observed in many host cells is reduced, i.e. the glycosylation pattern of the recombinant protein of interest is substantially more similar to the glycosylation pattern in its natural host than for example to the glycosylation pattern following production in S. cerevisiae in the absence of the overproduced CaM kinase.

In a preferred embodiment of this method, the nucleic acid coding for a polypeptide having the biological activity of a CaM kinase is selected from the following group:

i) a nucleic acid coding for CaM kinase from Saccharomyces cerevisiae (CMK2);

ii) a nucleic acid coding for a homologue of a CaM kinase having the biological activity of a CaM kinase;

iii) a nucleic acid derived from the nucleic acid set forth in i) or ii) by degeneration of the genetic code, by deletion, insertion, addition and/or nucleotide exchange, said nucleic acid having the biological activity of a CaM kinase;

iv) a fragment of one of the nucleic acids set forth in i) to iii), said fragment coding for a polypeptide having the biological activity of a CaM kinase;

v) a nucleic acid capable of hybridizing with a sequence complementary to the nucleic acids set forth in i) to iv).

Suitable host cells in which the recombinant proteins of the methods described above may be produced are plant cells, animal cells, yeast cells, fungal cells, or slime fungus cells.

In a preferred embodiment the animal cells are mammalian or insect cells.

Cells of the genera Saccharomyces, Schizosaccharomyces, Kluyveromyces, Hansenula, Pichia, Schwanniomyces, Candida and Yarrowia such as Pichia pastoris, Pichia pinus Kluyveromyces lactis. Hansenula polymorpha and Candida boidinii are preferred yeast cells. Preferred fungal cells according to the invention belong to the genera Aspergillus, e.g. Aspergillus niger, Neurospora, Rhizopus and Trichoderma. A slime fungus cell particularly preferred as an expression host is that of the genus Dictyostelium.

In a further preferred embodiment of the present invention, the genes for the polypeptide and the protein having the biological activity of elF4E and/or CaM kinase are expressed under the control of a promoter P1, which is a strong promoter.

An inducible promoter P1 is particularly preferred. An example of such a promoter is the GAL1 promoter (Johnston & Davis, 1984), which can be regulated by the two sugars glucose and galactose. Should the medium in or upon which the host cells are growing contain glucose, the promoter activity of genes under the control of the GAL1 promoter is induced, resulting in a relatively large quantity of mRNA being produced as the transcription product. By contrast, promoter activity is very low in the presence of galactose in the medium, which means that only relatively few mRNA molecules are produced.

In one embodiment of the present invention, the secretory protein is the enzyme phytase. Phytase is a plant enzyme which separates myo-inositol from phytinic acid. Owing to its lipotrophic effects, myo-inositol is employed for medicinal purposes in liver therapy. When phytase is added to the feed of monogastric animals (e.g. poultry and pigs), it enables them to absorb phosphate effectively from the food, and at the same time reduces the quantity of phosphate excreted. This means that little or no phosphate need be added artificially to the feed of animals with low-phosphate diets, and also that the resulting manure is less harmful to the environment owing to its reduced phosphate content.

In further embodiments, amylolytic enzymes, such as α-amylase or glucoamylase, or hormones, are produced. In preferred embodiments of the invention, the recombinant protein is overproduced. As already described above, production may be influenced by a number of factors, such as the vector employed, the selection system and the promoter. According to the invention, production is preferably placed under the control of strong and/or inducible promoters.

A further aspect of the present invention is the use of at least one nucleic acid to increase secretion of a recombinant secretory protein from a host cell, said nucleic acid being selected from the following group:

i) a nucleic acid coding for elF4E from Saccharomyces cerevisiae (CDC33);

ii) a nucleic acid coding for a homologue of elF4E having the biological activity of elF4E;

iii) a nucleic acid derived from the nucleic acid set forth in i) or ii) by degeneration of the genetic code, by deletion, insertion, addition and/or nucleotide exchange, said nucleic acid coding for a polypeptide having the biological activity of elF4E;

iv) a fragment of one of the nucleic acids set forth in i) to iii), said fragment coding for a polypeptide having the biological activity of an elF4E;

v) a nucleic acid capable of hybridizing with a sequence complementary to the nucleic acid sequences set forth in i) to iv);

vi) a nucleic acid coding for CaM kinase from Saccharomyces cerevisiae (CMK2);

vii) a nucleic acid coding for a homologue of a CaM kinase having the biological activity of a CaM kinase;

viii) a nucleic acid derived from the nucleic acid set forth in vi) or vii) by degeneration of the genetic code, by deletion, insertion, addition and/or nucleotide exchange, said nucleic acid coding for a polypeptide with the biological activity of a CaM kinase;

ix) a fragment of one of the nucleic acids set forth in vi) to viii), said fragment coding for a polypeptide having the biological activity of a CaM kinase;

x) a nucleic acid capable of hybridizing with a sequence complementary to the nucleic acids set forth in vi) to ix).

The coexpression of one of the nucleic acids listed above with the nucleic acid coding for a desired protein permits efficient secretion of the desired protein.

Recombinant proteins which are secreted from host organisms containing one or more of the above nucleic acids have the benefit that the desired protein is secreted more efficiently and can therefore be purified more easily. This reduces the production costs. Proteins thus obtained may be used for example as medicaments or food substitutes, or in the cosmetics industry.

In a preferred embodiment of the invention the above nucleic acids (i) to (x) are integrated into a vector. In this case the vector should contain polyadenylation and translation termination signals in addition to a promoter P1 functionally linked to the nucleic acid concerned.

In addition to the above methods, the present invention further provides a method for identifying a nucleic acid molecule coding for a protein permitting improved secretion of a recombinant secretory protein from a eukaryotic cell, said method comprising the following steps:

a) providing recombinant host cells which contain a nucleic acid coding for a secretory marker protein and being functionally linked to a promoter P3, with the growth of said host cells being inhibited under production of the secretory marker protein, the recombinant host cells further containing an expression vector in which DNA fragments from any given organism are under the control of a suitable promoter P1;

b) growing the recombinant host cells under conditions permitting selection for the presence of the expression vector;

c) selecting colonies exhibiting growth derepression;

d) analyzing the DNA contained in the expression vector from the given organism from the colonies exhibiting growth derepression;

e) identifying the nucleic acid sequences which permit derepression of growth inhibition following expression in the recombinant host cell.

The promoter P3 herein is any given promoter permitting expression of the gene for the secretory marker protein in the host cell concerned. The selection of a strong promoter may be advantageous herein.

The screening method according to the invention is based upon the finding that extreme overexpression of a suitable nucleic acid coding for a secretory protein causes overloading of the cell apparatus responsible for secretion. As a result, the endoplasmic reticulum and the Golgi apparatus are “clogged” by recombinant protein, and cell growth and protein secretion are arrested. Should a further recombinant protein capable of overcoming the bottleneck arising during secretion now be produced in such a cell, the cell resumes growth and division. By the testing of as a large a number of different recombinant proteins as possible, the method described here for the first time permits identification of proteins and the nucleic acid sequences encoding same which are capable of preventing or reversing a cell growth arrest.

The host cells employed in the above method may be plant cells, animal cells, yeast cells, fungal cells, or slime fungus cells. Mammalian and insect cells are preferred animal cells, and cells of the genera Saccharomyces, Schizosaccharomyces, Kluyveromyces, Hansenula, Pichia, Schwanniomyces, Candida and Yarrowia are preferred yeast cells. Particularly preferred yeast cells are those of the genus Hansenula polymorpha. Preferred fungal cells according to the invention are cells of the genera Aspergillus, e.g. Aspergillus niger, Neurospora, Rhizopus and Trichoderma. Furthermore, a cell of the genus Dictyostelium is included as a particularly preferred slime fungus cell for performance of the above method.

In one embodiment of this method, the promoter controlling production of the marker protein is an inducible promoter, preferably a strong promoter. The PDC1 promoter is an example of an inducible promoter. It is recommended that the recombinant host cells in step b) of the method be cultivated under induced conditions, i.e. when using an expression vector with PDC1 promoter, that the host cells are grown in a medium containing glucose in the absence of galactose.

In a further embodiment, the nucleic acid coding for a secretory marker protein is contained in a plasmid with a high number of copies per host cell. This may be achieved for example by the presence of the LEU2d gene.

According to the invention, the DNA fragments employed in step a) of the above method should preferably be derived from a cDNA gene bank. A cDNA gene bank from S. cerevisiae, e.g. that described by Liu et al. (1992), which was established in the basic vector pRS316 (CEN/ARS plasmid) (Sikorski and Hieter, 1989), is particularly preferred in this case.

In a further embodiment the DNA fragments employed in step a) may also be derived from a genomic gene bank, e.g. a gene bank from the S. cerevisiae genome.

In a particularly preferred embodiment, the expression vector in step a) containing DNA fragments from any given organism is the CEN/ARS vector pRS316 (Sikorski and Hieter, 1989).

Particular preference shall further be given to the enzyme glucoamylase (EC 3.2.1.3; glucoamylase with debranching activity) as the secretory marker protein (Dohmen, 1990). In a particularly preferred embodiment the nucleic acid sequence coding for glucoamylase in the above method is the GAM1 sequence from S. occidentalis.

The present invention further comprises the use of a recombinant host cell, said host cell containing a nucleic acid coding for a secretory marker protein and being under the control of a promoter, for identifying nucleic acid sequences which permit a derepression of growth inhibition following expression, the growth of the host cell being inhibited under expression of the genes for the marker protein.

The present invention further comprises a host cell which contains in its chromosome a nucleic acid introduced into the cell by a recombinant process, said nucleic acid being selected from the following group:

i) a nucleic acid coding for elF4E from Saccharomyces cerevisiae (CDC33);

ii) a nucleic acid coding for a homologue of elF4E having the biological activity of elF4E;

iii) a nucleic acid derived from the nucleic acid set forth in i) or ii) by degeneration of the genetic code, by deletion, insertion, addition and/or nucleotide exchange, said nucleic acid coding for a polypeptide having the biological activity of elF4E;

iv) a fragment of one of the nucleic acids set forth in i) to iii), said fragment coding for a polypeptide having the biological activity of an elF4E;

v) a nucleic acid capable of hybridizing with a sequence complementary to the nucleic acids set forth in i) and iv);

vi) a nucleic acid coding for CaM kinase from Saccharomyces cerevisiae (CMK2);

vii) a nucleic acid coding for a homologue of a CaM kinase having the biological activity of a CaM kinase;

viii) a nucleic acid derived from the nucleic acid set forth in vi) or vii) by degeneration of the genetic code, by deletion, insertion, addition and/or nucleotide exchange, said nucleic acid coding for a polypeptide having the biological activity of a CaM kinase;

ix) a fragment of one of the nucleic acids set forth in vi) to viii), said fragment coding for a polypeptide having the biological activity of a CaM kinase;

x) a nucleic acid capable of hybridizing with a sequence complementary to the nucleic acids set forth in vi) to ix).

The invention further comprises a kit containing a host cell suitable for the secretion of proteins, and an expression vector which comprises a nucleic acid being functionally linked to a promoter and coding for a polypeptide having the biological activity of elF4E, said nucleic acid being selected from the following group:

i) a nucleic acid coding for elF4E from Saccharomyces cerevisiae (CDC33);

ii) a nucleic acid coding for a homologue of elF4E having the biological activity of elF4E;

iii) a nucleic acid derived from the nucleic acid set forth in i) or ii) by degeneration of the genetic code, by deletion, insertion, addition and/or nucleotide exchange, said nucleic acid coding for a polypeptide having the biological activity of elF4E;

iv) a fragment of one of the nucleic acids set forth in i) to iii), said fragment coding for a polypeptide having the biological activity of an elF4E;

v) a nucleic acid capable of hybridizing with a sequence complementary to the nucleic acids set forth in i) to iv).

The expression vector may be present episomally within the host cell or provided as a separate preparation.

The use of this kit is intended to enable the user to effect, efficiently and easily, secretion of a protein of interest from a eukaryotic host cell, in order to be able to obtain it faster and in greater quantities than was possible in the past.

The invention further comprises a kit containing a host cell suitable for the secretion and/or glycosylation of proteins, and an expression vector which comprises a nucleic acid being functionally linked to a promoter and coding for a polypeptide with the biological activity of a CaM kinase, said nucleic acid being selected from the following group:

i) a nucleic acid coding for CaM kinase from Saccharomyces cerevisiae (CMK2);

ii) a nucleic acid coding for a homologue of a CaM kinase having the biological activity of a CaM kinase;

iii) a nucleic acid derived from the nucleic acid set forth in i) or ii) by degeneration of the genetic code, by deletion, insertion, addition and/or nucleotide exchange, said nucleic acid coding for a polypeptide having the biological activity of a CaM kinase;

iv) a fragment of one of the nucleic acids set forth in i) to iii), said fragment coding for a fragment having the biological activity of a CaM kinase;

v) a nucleic acid capable of hybridizing with a sequence complementary to the nucleic acids set forth in i) to iv).

This kit offers the user the benefit that, where a protein is secretory, he can attain at one and the same time not only efficient secretion of the protein, but also a glycosylation of the recombinant protein which strongly resembles the glycosylation pattern of the protein in the natural eukaryotic host cell, i.e. the otherwise frequently occurring hyperglycosylation is avoided or reduced.

The invention further comprises a kit containing a host cell suitable for the secretion and/or glycosylation of proteins, and an expression vector which comprises nucleic acids being functionally linked to the corresponding promoter and coding for polypeptides having the biological activity of a CaM kinase and an elF4E, said nucleic acids being selected from the following group:

i) a nucleic acid coding for elF4E from Saccharomyces cerevisiae (CDC33);

ii) a nucleic acid coding for a homologue of elF4E having the biological activity of elF4E;

iii) a nucleic acid derived from the nucleic acid set forth in i) or ii) by degeneration of the genetic code, by deletion, insertion, addition and/or nucleotide exchange, said nucleic acid coding for a polypeptide having the biological activity of elF4E;

iv) a fragment of one of the nucleic acids set forth in i) to iii), said fragment coding for a polypeptide having the biological activity of an elF4E;

v) a nucleic acid sequence capable of hybridizing with a sequence complementary to the nucleic acids set forth in i) to iv).

vi) a nucleic acid coding for CaM kinase from Saccharomyces cerevisiae (CMK2);

vii) a nucleic acid coding for a homologue of a CaM kinase having the biological activity of a CaM kinase;

viii) a nucleic acid derived from the nucleic acid set forth in vi) or vii) by degeneration of the genetic code, by deletion, insertion, addition and/or nucleotide exchange, said nucleic acid coding for a polypeptide having the biological activity of a CaM kinase;

ix) a fragment of one of the nucleic acids set forth in vi) to viii), said fragment coding for a polypeptide having the biological activity of a CaM kinase;

x) a nucleic acid capable of hybridizing with a sequence complementary to the nucleic acids set forth in vi) to ix).

In a further embodiment of the present invention, a kit according to the invention further contains an empty expression vector suitable for the cloning of a nucleic acid coding for a recombinant and/or recombinant secretable protein. The contents of this kit are intended for ease of use by a user wishing to express a particular nucleic acid quickly and reliably in a suitable host in order to attain efficient secretion and/or reduced hyperglycosylation of the recombinant protein concerned.

The present invention further provides a kit containing a host cell as described above carrying the sequences required for increasing secretion not on a vector, but in its genome, together with an empty expression vector suitable for the cloning of a nucleic acid coding for a recombinant and/or recombinant secretory protein. This kit enables production and where required secretion of a protein of interest to be accomplished effectively by transformation of the host cell likewise contained in the kit with the expression vector.

The figures and examples below are intended to describe and illustrate the various aspects of the present invention. The selected genetic components and DNA sequences employed are intended to demonstrate the essential principles of the invention. The examples provided should not therefore be deemed restrictive.

### FIGURES

FIG. 1 shows a plasmid map of plasmid pMF23. The plasmid is an E. coli/S. cerevisiae shuttle vector. It carries the following functional units: an origin of replication (oriR) and the β-lactamase gene (bla) for propagation and selection in E. coli, and the entire 2μ DNA for propagation in S. cerevisiae. Marker sequences for selection in yeast are the TRP1 gene and the LEU2d allele. The plasmid pMF23 contains the GAM1 gene from S. occidentalis including the terminator, the GAM1 gene being under the control of the PDC1 promoter originating from S. cerevisiae.

pMF25 shares the structure of pMF23 and possesses essentially the same coding sequence, but contains a C-terminal haemagglutinin epitope from an influenza virus.

FIG. 2 shows the subcellular localization of Gam1p protein in various strains of S. cerevisiae as a function of the expression strength. The GAM1 gene was expressed either from the “single copy plasmid” pMF28 or the “multi-copy plasmid” pMF25. The pMF28 plasmid is a CEN vector containing the URA3⁺ gene as a selection marker and the GAM1 sequence fused with a sequence coding for the haemagglutinin. The cells were grown in 100 ml minimal medium, without uracil (pMF28) or without tryptophane (pMF25), with galactose or glucose (as indicated) as the sole carbon source, or were arrested in their growth for 30 hours in a leucine-free minimal medium. Cell fractions were separated by centrifugation with a linear sucrose gradient (7-47%), and their Gam1p protein content analyzed by SDS-PAGE and Western blotting with specific anti-haemagglutinin antibodies. The subcellular compartments were identified by the presence of specific marker proteins. Dolichol phosphate mannose synthase (Dpm1p—endoplasmic reticulum), oligosaccharyl transferase (Och1p—early Golgi), ATPase (Pmr1p—medial Golgi), and endoprotease (Kex2p—late Golgi) were employed as the marker proteins.

FIG. 3 shows the Gam1p activity determined in the culture supernatant and the cell wall fraction of S. cerevisiae strains. The cells were used to inoculate 20 ml SG medium without tryptophane or without tryptophane and without leucine. Supertransformants also containing plasmid with the DNA coding for CDC33 were cultivated without uracil. The cells were grown until they reached the stationary phase, after which the glucoamylase activity was determined as described in Example 3. The activity values determined are the averages of at least three independent measurements.

FIG. 4 shows the determination of Gam1p activity determined in the culture supernatant and the cell wall fraction of S. cerevisiae strains. All conditions were identical to those shown in FIG. 3. A strain supertransformed with the plasmid coding for CMK2 served as a comparison.

FIG. 5 shows the degree of glycosylation of Gam1p in strains of S. cerevisiae overexpressing CMK2 and CDC33. The strains were cultivated as described in FIG. 2, and subcellular fractions were tested for their Gam1p content. In strains overexpressing CMK2, a large proportion of the Gam1p protein is not hyperglycosylated.

FIG. 6 shows the degree of glycosylation of phytase in strains of Hansenula polymorpha overexpressing CMK2. H. polymorpha cells producing phytase were supertransformed with a plasmid containing the CMK2 gene originating from S. cerevisiae under the control of the GAL1 promoter from S. cerevisiae. The strain producing phytase and representative examples of the supertransformants were cultivated in glycerine medium (5% glycerine, 0.1 M P0₄, pH 5.0). The secreted phytase protein was analyzed by SDS-PAGE and compared with the secreted product deglycosylated by treatment with Endo H. The figure shows phytase protein secreted by the original phytase production strain compared with phytase produced by a supertransformed strain which in addition carries a CMK2 construct with the GAL1 promoter. The degree of hyperglycosylation is reduced in the supertransformants, enabling distinct polypeptide bands to be observed.

Lanes 2, 4, 6, 8, 10 correspond to phytase samples treated with Endo H; the samples of lanes 1, 3, 5, 7 and 9 were not pretreated. The following were applied to lanes

1 to 4: phytase secreted by supertransformed cells (two culture supernatants); to lanes

5 to 10: phytase secreted by the original production strain (three culture supernatants); to lane

11: a molecular weight standard: the upper band corresponds to 66.3 kDa, the lower band to 55.4 kDa.

FIG. 7 shows the plasmid features of a host cell suitable for efficient secretion or reduction in hyperglycosylation of a recombinant protein of interest (A) and of a host cell suitable for identification of nucleic acid molecules capable of derepressing growth inhibition of the host (B).

Materials and methods
Saccharomyces cerevisiae strains

| Strain | Genotype | Reference |
|---|---|---|
| JD52 | MATα, leu2-3, 112, trp1-Δ63, ura3-52, | Dohmen et al., 1995 |
| | his3-Δ200, lys2-801, cir⁺ | |
| MF9 | MATα, leu2-Δ1100, trp1-Δ63, ura3-52, | See below |
| | his3-Δ200, lys2-801, cir° | |

In the JD52 strain of S. cerevisiae, the ORF of LEU2 was restored by homologous recombination with a functional LEU2 fragment. The functional LEU2 fragment was obtained from pUC/LEU#5 as a SalI-Xhol fragment. The restored LEU2 gene was then deleted in the region from −407 to +748. The endogenous 2μ DNA was then removed from the strain by the method described by Erhart and Hollenberg (1 983), thus giving rise to the MF9 strain.

Plasmids

| Plasmid | Construction, DNA sequences contained | Reference |
|---|---|---|
| pJDcPG-15 | CEN/ARS plasmid, contains the GAM1 | Dohmen, 1989 |
| | expression cassette, URA3 | |
| pJDaG-15 | TRP1/ARS plasmid, contains the GAM1 | Dohmen, 1989 |
| | expression cassette | |
| pJDB219 | Complete 2μ LEU2d E. coli shuttle vector | Beggs, 1981 |
| pRS316 | CEN/ARS, URA3 | Sikorski and Hieter, 1989 |
| pUC19 | E. coli cloning vector | Sambrook et al., 1989 |
| pUC/LEU#5 | pUC19 with a genomic Sa/I-Xhol fragment | J. Dohmen, not published |
| | containing the LEU2 gene | |
| pMF23 | The ARS element was removed from | This application, |
| | pJDaG-15 by digesting with Bg/II-Pstl. The | 2μ TRP1/LEU2d |
| | cohesive ends were filled in by treatment with | expression plasmid for |
| | T4 polymerase, and the plasmid was religated. | GAM1 under the control |
| | The 2μ LEU2d components from pJDB219, | of the P_{PDC1} promoter |
| | obtained by partial digestion of the plasmid with | |
| | EcoRI, were ligated into the EcoRI restriction | |
| | site. | |
| pMF25 | The PCR products MF238/246 (GAM1) and | This application, as |
| | MF247/248 (GAM1 terminator) were digested | pMF23; codes for a |
| | with Sacl-Kpnl and Xbal-Notl, respectively. The | glucoamylase having a |
| | HA₂ epitope was obtained as a Kpnl-Xbal | double HA epitope on the |
| | fragment from pJD307 (Dohmen et al., 1995) | C-terminus |
| | and ligated with a Sacl-Kpnl GAM1 fragment | |
| | and a Xbal-Notl GAM1 terminator fragment into | |
| | the pMF23 plasmid opened with Sacl/Notl. | |
| pMF28 | Recloning of the Sacl/Notl fragment from | This application, “single- |
| | pMF25 into pJDcPG-15 | copy” expression plasmid |
| | | for GAM1 |

### METHODS

### Media

Media for Saccharomyces cerevisiae:

| | |
|---|---|
| YPD full medium | 1% yeast extract; 2% peptone; 2% glucose |
| YPGal full medium | 1% yeast extract; 2% peptone; 2% galactose |
| SD minimal medium | 6.7 g/l yeast nitrogen base w/o amino acids; 2% glucose; |
| | 50 mM citrate pH 4.5 |
| The following were added, | Histidine and methionine 0.01 g/l each; arginine 0.02 g/l; |
| depending on the selection | lysin and tryptophane 0.04 g/l; threonine 0.05 g/l; leucine, |
| | isoleucine and phenylalanine 0.06 g/l; uracil 40 mg/l, |
| | adenine 20 mg/l |
| SG minimal medium | As SD, but with 2% galactose in place of 2% glucose |

Solid culture media contained an additional 1.8% agar; in some cases 0.5% soluble starch was added according to Zulkowsky (Merck). The yeasts were grown at 30° C. unless indicated otherwise.

Media for Escherichia coli:

| | |
|---|---|
| LB medium | 1% tryptone; 0.5% yeast extract; 1% NaCl |
| SOC medium | 2% tryptone, 0.5% yeast extract; 20 mM |
| | glucose, 10 mM NaCl; 2.5 mM KCl; |
| | 10 mM MgCl₂; 10 mM MgSO₄; pH 7.5 |
| M9 minimal medium | Na₂HPO₄ × 7 H₂O 12.8 g/l, KH₂PO₄ 3 g/l, |
| | NaCl 0.5 g/l, NH₄Cl 1 g/l |

Solid culture media contained an additional 1.8% agar. 120 mg/l ampicillin was added to the medium for selection of cells containing plasmid. The bacteria were cultivated at a temperature of 37° C.

### EXAMPLE 1

### Preparation of a System for the Identification of Genes Influencing the Secretion of Heterologous Proteins in an S. cerevisiae Host Cell

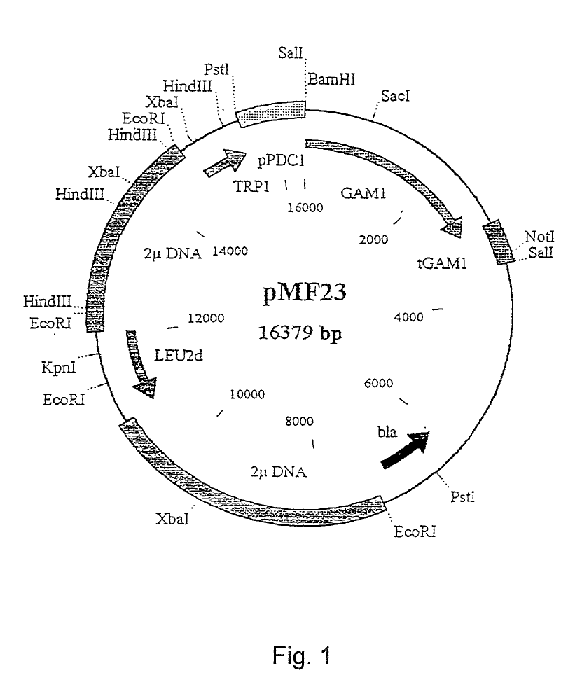

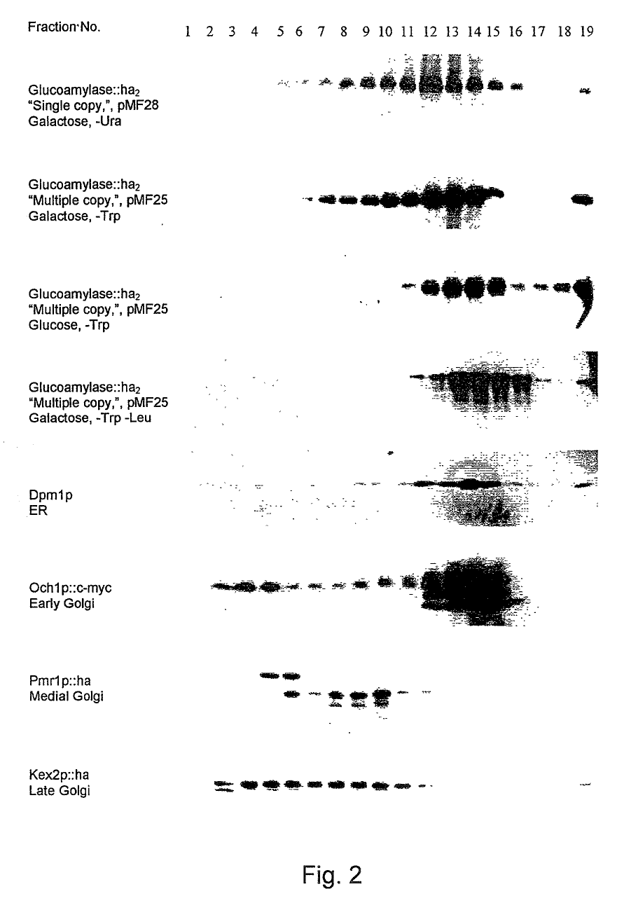

A 2μ plasmid derivative denoted pMF23 was prepared from the plasmids pJDaG-15 and pJDB219 (Dohmen, 1989; Beggs, 1981). The resulting plasmid is shown in FIG. 1 and contains, in addition to the complete 2μ DNA, a GAM1 sequence originating from S. occidentalis which is fused to a PDC1 promoter element. The plasmid also contains the selection markers TRP1 and LEU2d. LEU2d is an allele of the LEU2 gene in which the major part of the promoter is deleted and which is therefore expressed weakly. The use of these two selection markers enables the selective pressure to be varied and the copy number of the plasmids introduced into the MF9 strain of S. cerevisiae (trp1, leu2, cir°) to be influenced. An extremely high copy number of the introduced plasmid is obtained under LEU2d selective conditions, whereas under selective conditions for TRP1 a copy number is obtained which though high, is nonetheless lower than that for LEU2d selection. Under LEU2d selective conditions, recombinant strains obtained following transformation with the plasmids described above thus contain a relatively high number of copies of the GAM1 gene which is transcriptionally regulated by the PDC1 promoter. Growth on medium containing glucose attains high PDC1 promoter activity, whereas growth on galactose leads to lower activity. Transformants grown under LEU2d selective conditions are incapable of growing on media containing either glucose or galactose. Comparison of these cells with transformants produced from control transformations in which plasmids coding for enzymatically inactive glucoamylase were employed revealed that the growth inhibition observed under LEU2d selective conditions was caused by the secretory stress being too high for the recombinant enzyme. The inhibition of growth was not caused by the enzymatic activity of the glucoamylase. This was confirmed by the observation that Gam1p production under less stringent conditions led to an accumulation of the enzyme in the endoplasmic reticulum and the cis-Golgi (FIG. 2).

Transformants of this kind were employed for retransformation with the cloned DNA sequences of the S. cerevisiae cDNA gene bank. A cDNA gene bank from S. cerevisiae was employed for this purpose which was cloned under the control of the GAL1 promoter into a CEN/ARS vector (Liu et al. 1992; Ausubel et al., 1987). The S. cerevisiae strains were grown either on non-selective medium (YPD) or on a minimal medium. Strains which had regained the ability to secrete the glucoamylase under the stringent conditions described above and then to grow had incorporated DNA fragments which led to their being able to overcome the observed inhibition of growth and thus the limitation of secretion. Plasmids from transformants which exhibited suppression of growth inhibition of the host strain overproducing Gam1p were analysed by DNA sequencing.

Analysis of the glucoamylase protein obtained under the conditions according to the invention and comparison of it with standard isolates, i.e. isolates obtained under identical conditions with the exception that corresponding empty vectors were employed in place of the vectors containing CMK2 and/or CDC33, further permitted assessment of secondary modifications, such as the glycosylation pattern of the secreted product.

### EXAMPLE 2

### Identification of S. cerevisiae Genes which Suppress the Growth Inhibition in S. cerevisiae Strains Overproducing Gam1p

Strain MF9: pMF23 was transformed with the cDNA gene bank described above. 420,000 transformants were grown for two days on SG plates without tryptophane and uracil. The colonies thus obtained were replicated by replica plating on SG plates without leucine, tryptophane and uracil. 320 leucine-prototrophic clones were obtained, 66 of which exhibited growth as a function of the carbon source (growth on galactose, no growth on glucose). All clones obtained secreted glucoamylase. The cDNA inserts of the 55 clones exhibiting the strongest growth were analyzed by PCR amplification. Eight different cDNA sizes could be distinguished, three of which were present several times. A total of 13 plasmids were isolated and analyzed by DNA sequencing. The ability of the isolated DNA sequences to derepress growth inhibition was confirmed by retransformation of the individual sequences into the strain MF9:pMF23. The identity of the sequences obtained was determined by comparison with data from sequence databases. Four instances of the CDC33 sequence were present among these DNA sequences, whereas the CMK2 sequence occurred three times. CDC33 codes an essential part of the “cap binding complex” by binding to the 5′ cap structure of mRNAs (Sonenberg, 1996). The secretion efficiency of Gam1p was compared to that of the host strain originally secreting Gam1p, and the secretory product tested for glycosylation.

### EXAMPLE 3

### Gam1p Production in Strains Overexpressing the CDC33 Gene from S. cerevisiae

The recombinant strain MF9:pMF23 was retransformed with the plasmid pCDC33, which contains the CDC33 coding sequence from S. cerevisiae under the control of the GAL1 promoter. The original strain and the retransformed strain were grown in SG medium either without tryptophane (TRP1 selection) or without tryptophane and without leucine (TRP1 and LEU2d selection) in 20 ml at 30° C. until the stationary phase was reached.

The glucoamylase activity secreted by the recombinant strains was measured exploiting the newly gained ability of the yeast cells to degrade starch. These cells were therefore placed on plates containing 0.5% soluble starch (according to Zulkowski, Merck, Darmstadt). Following cell growth, the plates were iodized with iodine crystals. The quantity of glucoamylase secreted was determined from the diameter of the colourless haloes on the red-coloured background. For more precise determination of the glucoamylase activities of different strains, the latter were grown in the medium described above, and the glucose formation from a starch substrate was measured (Dohmen et al., 1990; Gellissen et al., 1991). The components of the “glucose test kit” (Merck, Darmstadt) were employed for glucose measurement in accordance with the manufacturer's instructions. Glucoamylase activity was measured in samples of the culture supernatant and cell wall fractions. The enzyme activity was also determined in whole cell extracts obtained from cells in the exponential growth phase. The activity was determined by at least three independent measurements in each case.

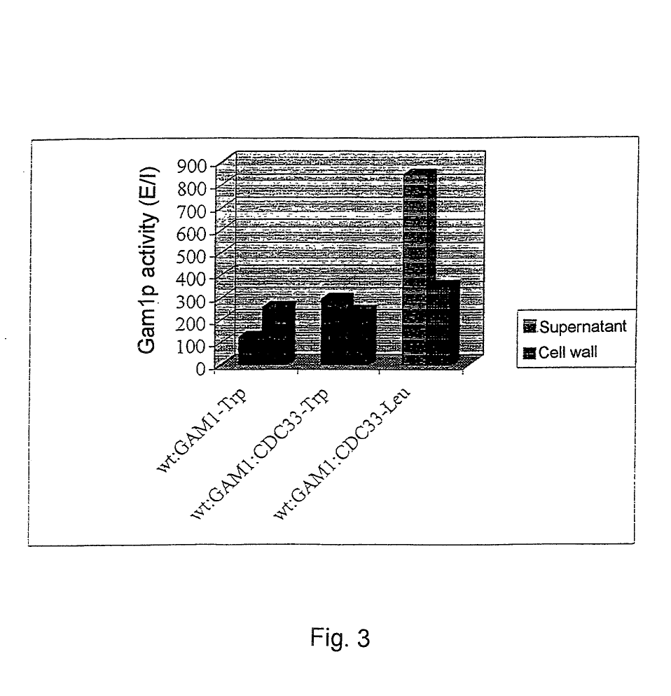

Overexpression of the CDC33 gene led to a 2.5-fold increase in the production of secreted glucoamylase in the case of TRP1 selection and a 7-fold increase in the case of LEU2d selection (see FIG. 3).

| | | | | | Activity in the |
|---|---|---|---|---|---|
| | | | Activity in | Activity in the | total cell extract |
| | | | supernatant | cell wall fraction | (units × 10⁻³/g |
| Strain | Selection | OD₆₀₀ | (units/l) | (units/l) | protein) |
| MF9:pMF23 | TRP1 | 5.0 | 122.0 ± 18.6 | 256.3 ± 14.9 | 54.7 ± 8.4 |
| MF9:pMF23 | TRP1 | 5.0 | 292.9 ± 34.5 | 237.1 ± 15.0 | 53.2 ± 2.5 |
| pCDC33 | | | | | |
| MF9:pMF23 | LEU2d | 4.5 | 841.8 ± 12.0 | 350.1 ± 27.7 | n.d. |
| pCDC33 | | | | | |

n.d.: not determined

### EXAMPLE 4

### Gam1p Production of Strains Overexpressing the S. cerevisiae CMK2 Gene

The recombinant strain MF9:pMF23 was retransformed as described above with the plasmid pCMK2, which contains the CMK2 coding sequence from S. cerevisiae under the control of the GAL1 promoter. The original strain and the retransformed strain were grown at 30° C. in 50 ml SG medium either without tryptophane (TRP1 selection) or without tryptophane and without leucine (TRP1 and LEU2d selection) until the stationary phase was reached. The glucoamylase activity was measured in samples of the culture supernatants and the cell wall fractions as described above. The enzyme activity was also determined in samples of whole cell extracts obtained from cells in the stationary phase. The activity was determined by at least three independent measurements.

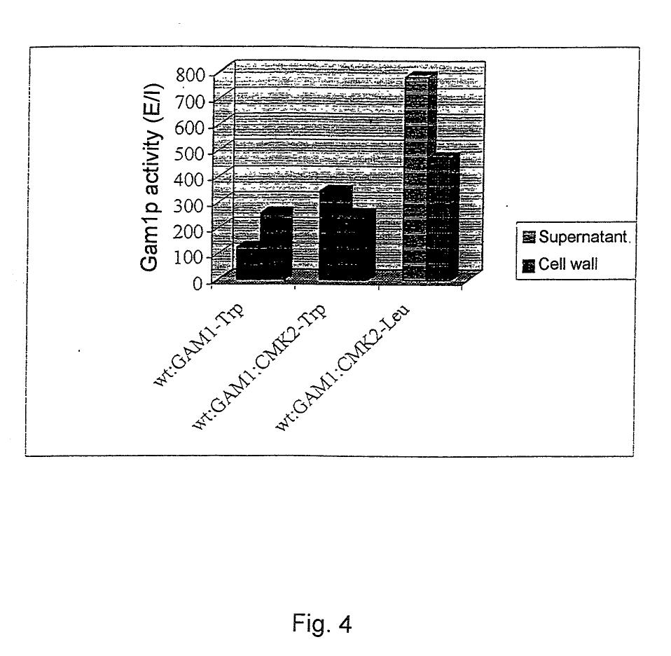

Overexpression of the CMK2 gene led to a three-fold increase in the production of secreted glucoamylase in the case of TRP1 selection and a 6.4-fold increase in the case of LEU2d selection (see FIG. 4).

| | | | | | Activity in the |
|---|---|---|---|---|---|
| | | | | | whole cell |
| | | | Activity in | Activity in the | extract |
| | | | supernatant | cell wall fraction | (units × 10⁻³/g |
| Strain | Selection | OD₆₀₀ | (units/l) | (units/l) | protein) |
| MF9:pMF23 | TRP1 | 5.0 | 122.0 ± 18.6 | 256.3 ± 14.9 | 54.7 ± 8.4 |
| MF9:pMF23 | TRP1 | 5.0 | 341.8 ± 5.0 | 254.4 ± 24.6 | 56.1 ± 15.3 |
| pCMK2 | | | | | |
| MF9:pMF23 | LEU2d | 4.5 | 781.4 ± 12.0 | 470.4 ± 19.7 | n.d. |
| pCMK2 | | | | | |

n.d.: not determined

### EXAMPLE 5

### Degree of Glycosylation of Gam1p in Recombinant S. cerevisiae Strains Overexpressing CDC33 and CMK2 Genes

The Gam1p protein is an enzyme with a molecular weight of >140 kDa. The calculated molecular weight of the amino acid sequence is 104 kDa. The difference between apparent and calculated molecular weight can be ascribed to the presence of N- and O-linked glycosylation. Treatment of Gam1p with the PNGase F enzyme, which specifically removes N-linked sugar units, leads to the molecular weight being reduced to 120 kDa. The remaining difference between 120 kDa and the calculated molecular weight of 104 kDa can be ascribed to O-linked sugar units. In this example, the Gam1p protein produced in the original S. cerevisiae strain is compared with the Gam1p protein produced with the CDC33 and CMK2 supertransformants. For this purpose, a glucoamylase with a C-terminal HA marker was prepared for immunological identification of the heterologous enzyme from cell fractions by means of Western Blot analysis. Cell extracts were prepared by gentle homogenization, and cell fractions obtained by centrifugation in a sucrose density gradient. Different cellular compartments were identified with the aid of marker enzymes specific to certain subcellular fractions (see FIG. 2). The presence and size of the heterologous Gam1p protein was determined by SDS-PAGE according to Laemmli (Laemmli, 1970). The separated polypeptides were transferred to PVDF membranes (Schleicher and Schuell) and treated with specific antibodies against the HA epitope (BabCo). In the strains which overexpressed the CMK2 gene, the Gam1p protein population consisted of molecules with a molecular weight of 140 kDa and 104 kDa. This shows that expression of the CMK2 gene contributes to the production of recombinant proteins the molecular weight of which corresponds to that of the unmodified amino acid chain. This effect was not observed in strains overexpressing CDC33.

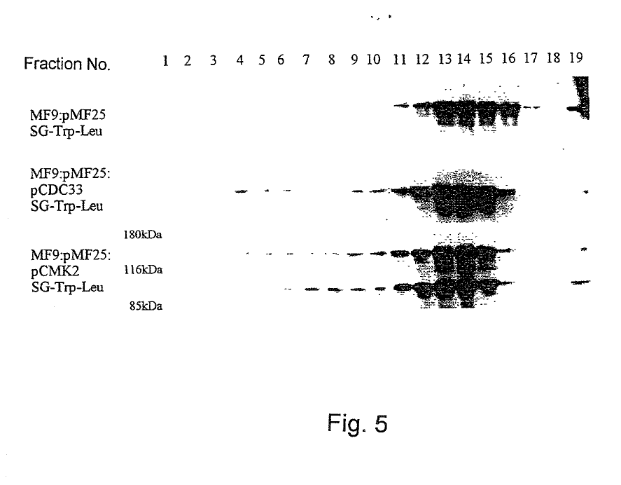

These results demonstrate that CMK2 overexpression leads to both an improvement of secretion, and a reduction in glycosylation. Conversely, overexpression of CDC33 results solely in an increase in secretion. The degree of glycosylation is not influenced in this case (see FIG. 5).

### EXAMPLE 6

### Degree of Glycosylation of a Recombinant Phytase in H. polymorpha Strains Overexpressinq the CMK2 Gene from S. cerevisiae

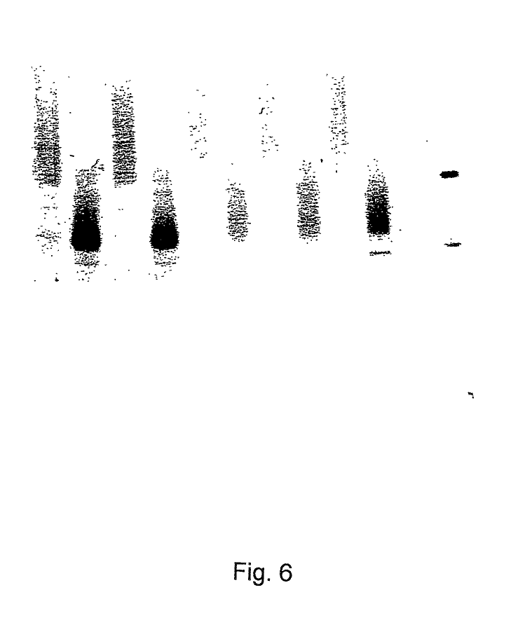

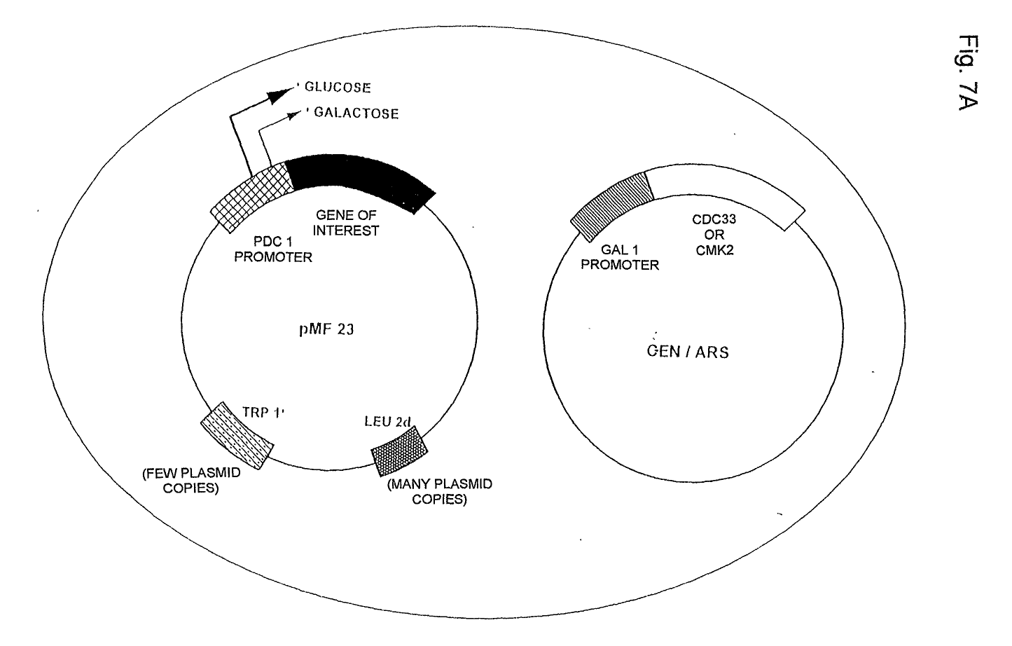

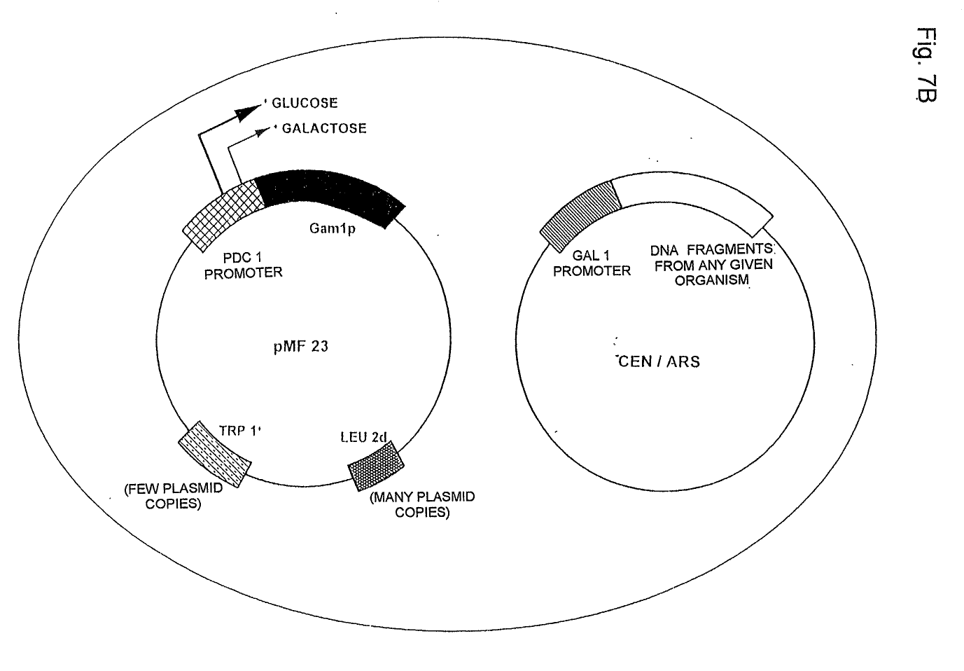

The influence of the CMK2 gene upon the glycosylation of heterologous proteins was studied in a phytase-producing H. polymorpha strain as described by Mayer et al., 1999. The strain contains 40 copies, integrated into the genome, of a phytase expression plasmid in which the coding sequence for phytase is fused to an FMD promoter element from H. polymorpha for expression control. The phytase variant concerned comprises an amino acid chain with a calculated molecular weight of 55 kDa. Hyperglycosylation results in proteins in a range between 60 kDa and 110 kDa. The production strain was supertransformed with a plasmid carrying the CMK2 gene from S. cerevisiae either under control of the GAL1 promoter also from S. cerevisiae, or under control of the FMD promoter from H. polymorpha. Examples of uses of the FMD promoter are published in EP 299108. The resulting supertransformants were studied by comparative SDS-PAGE to establish the degree of glycosylation of the secreted phytase. In both cases, hyperglycosylation was found to be reduced. Instead of a number of protein bands with different molecular weights, bands with distinct sizes appeared (FIG. 6).

Cited Literature:

Altmann and Trachsel, Nuc. Acid Res. 17, 5923 (1989)

Ausubel et al. (eds), Current protocols in molecular biology.

Wiley & Sons, New York (1987)

Beggs, Molecular genetics in yeast, von Wettstein et al. eds., Munksgaard, Copenhagen (1981)

Brenner et al., Mol. Cell. Biol. 8, 3556 (1988)

Dohmen, PhD thesis, Heinrich-Heine-Universität Düsseldorf (1989)

Dohmen et al., Gene 95, 111 (1990)

Dohmen et al., J. Biol. Chem. 270, 18099 (1995)

Erhart and Hollenberg, J. Bact. 156, 625 (1983),

Gellissen et al., Biotechnology 9, 291 (1991)

Gellissen and Hollenberg, Gene 190, 87 (1997)

Giga-Hama and Kumagai, “Foreign gene expression in fission yeast Schizosaccharomyces pombe”, Springer, Berlin (1997)

Hinnen et al., Gene expression in recombinant microorganisms, Smith, ed., Marcel Dekker, N.Y., 121 (1994)

Hollenberg and Gellissen, Curr. Opin. Biotechnol. 8, 554 (1997)

Johnston & Davis, Mol. Cell. Biol. 4 (8), 1440 (1984)

Laemmli U, Nature 227, 680 (1970)

Liu et al., Genetics 132, 665 (1992)

Mayer et al., Biotechnol. Bioeng. 63, pp. 373-381 (1999)

Ohya et al., J. Biol. Chem. 266, 12784 (1991)

Pausch et al., EMBO J. 10, 1511 (1991)

Pearson and Lipman, Proc. Natl. Acad. Sci. USA 85, 2444 (1988)

Sakei et al., Genetics 129, 499 (1988)

Sambrook, Fritsch & Maniatis, Molecular Cloning—A Laboratory Handbook Cold Spring Harbor Laboratory Press (1989)

Shulman, Curr. Opin. Cell Biol. 5, 247 (1993)

Shusta et al., Nature/Biotechnology 116, 773 (1998)

Sikorski & Hieter, Genetics 122, 19 (1989)

Smith et al., Science 229, 1219 (1985)

Sonenberg, Translational control, Mathews et al. eds, Cold Spring Harbour, N.Y. (1996)

WO98/13473

1. Method for producing a recombinant secretory protein, comprising the following steps:
a) expressing a nucleic acid coding for the secretory protein in a suitable host cell, together with a nucleic acid coding for a polypeptide having the biological activity of the eukaryotic translation initiation factor 4E (elF4E) and/or for a polypeptide having the biological activity of a Ca²⁺/calmodulin-dependent protein kinase (CaM kinase), said nucleic acid coding for a polypeptide having the biological activity of elF4E or CaM kinase being under the control of a promoter P1;
b) secreting the protein from the cell, and
c) obtaining the secreted protein.

2. Method according to claim 1, characterized in that the nucleic acid coding for a polypeptide having the biological activity of elF4E is selected from the following group:
i) a nucleic acid coding for elF4E from Saccharomyces cerevisiae (CDC33);
ii) a nucleic acid coding for a homologue of elF4E having the biological activity of elF4E,
iii) a nucleic acid derived from the nucleic acid set forth in i) or ii) by degeneration of the genetic code, by deletion, insertion, addition and/or nucleotide exchange, said nucleic acid coding for a polypeptide having the biological activity of elF4E;
iv) a fragment of one of the nucleic acids set forth in i) to iii), said fragment coding for a polypeptide having the biological activity of an elF4E;
v) a nucleic acid capable of hybridizing with a sequence complementary to the nucleic acids set forth in i) to iv).

3. Method for producing a recombinant protein, comprising the following steps:
a) expressing a nucleic acid coding for the recombinant protein in a suitable host cell, together with a nucleic acid coding for a polypeptide having the biological activity of a Ca²⁺/calmodulin-dependent protein kinase (CaM kinase), said nucleic acid coding for a polypeptide having the biological activity of CaM kinase being under the control of a promoter P1;
b) obtaining the protein.

4. Method according to at least one of claims 1 to 3, characterized in that the nucleic acid coding for a polypeptide having the biological activity of a CaM kinase is selected from the following group:
i) a nucleic acid coding for CaM kinase from Saccharomyces cerevisiae (CMK2);
ii) a nucleic acid coding for a homologue of a CaM kinase having the biological activity of a CaM kinase;
iii) a nucleic acid derived from the nucleic acid set forth in i) or ii) by degeneration of the genetic code, by deletion, insertion, addition and/or nucleotide exchange, said nucleic acid having the biological activity of a CaM kinase;
iv) a fragment of one of the nucleic acids set forth in i) to iii), said fragment coding for a polypeptide having the biological activity of a CaM kinase;
v) a nucleic acid capable of hybridizing with a sequence complementary to the nucleic acids set forth in i) to iv).

5. Method according to at least one of claims 1 to 4, characterized in that the suitable host cell is a plant cell, animal cell, yeast cell, fungal cell or slime fungus cell.

6. Method according to at least one of claims 1 to 5, characterized in that the animal cell is a mammalian cell or insect cell.

7. Method according to at least one of claims 1 to 6, characterized in that the yeast cell is a cell of the genus Saccharomyces, Schizosaccharomyces, Kluyveromyces, Hansenula, Pichia, Schwanniomyces, Candida or Yarrowia.

8. Method according to at least one of claims 1 to 7, characterized in that the fungal cell is a cell of the genus Aspergillus, Neurospora, Rhizopus or Trichoderma.

9. Method according to at least one of claims 1 to 8, characterized in that the slime fungus cell is a cell of the genus Dictyostelium.

10. Method according to at least one of claims 1 to 9, characterized in that the promoter P1 is a strong promoter.

11. Method according to one of claims 1 to 10, characterized in that the promoter is inducible.

12. Method according to at least one of claims 1 to 11, characterized in that the secretory protein is selected from the following group: phytase, glucoamylase, phosphatase, growth factors.

13. Use of at least one nucleic acid to increase secretion of a recombinant secretory protein from a host cell, characterized in that the nucleic acid is selected from the following group:
i) a nucleic acid coding for elF4E from Saccharomyces cerevisiae (CDC33);
ii) a nucleic acid coding for a homologue of elF4E having the biological activity of elF4E;
iii) a nucleic acid derived from the nucleic acid set forth in i) or ii) by degeneration of the genetic code, by deletion, insertion, addition and/or nucleotide exchange, said nucleic acid coding for a polypeptide having the biological activity of elF4E;
iv) a fragment of one of the nucleic acids set forth in i) to iii), said fragment coding for a polypeptide having the biological activity of an elF4E;
v) a nucleic acid sequence capable of hybridizing with a sequence complementary to the nucleic acid sequences set forth in i) to iv);
vi) a nucleic acid coding for CaM kinase from Saccharomyces cerevisiae (CMK2);
vii) a nucleic acid coding for a homologue of a CaM kinase having the biological activity of a CaM kinase;
viii) a nucleic acid derived from the nucleic acid set forth in vi) or vii) by degeneration of the genetic code, by deletion, insertion, addition and/or nucleotide exchange, said nucleic acid coding for a polypeptide having the biological activity of a CaM kinase;
ix) a fragment of one of the nucleic acids set forth in vi) to viii), said fragment coding for a polypeptide having the biological activity of a CaM kinase;
x) a nucleic acid capable of hybridizing with a sequence complementary to the nucleic acids set forth in vi) to ix).

14. Use of a nucleic acid according to claim 13, characterized in that the nucleic acid is integrated into a vector.

15. Method for identifying a nucleic acid sequence coding for a protein permitting improved secretion of a recombinant secretory protein from a eukaryotic cell, said method comprising the steps:
a) providing recombinant host cells which contain a nucleic acid coding for a secretory marker protein and being under the control of and functionally linked to a promoter P2, with the growth of the host cell being inhibited under expression of the gene for the secretory marker protein, the recombinant host cells further containing an expression vector in which DNA fragments from any given organism are under the control of a suitable promoter;
b) growing the recombinant host cells under conditions permitting selection for the presence of the expression vector;
c) selecting colonies exhibiting growth derepression;
d) analyzing the DNA contained in the expression vector from the given organism from the colonies exhibiting growth derepression;
e) identifying the, nucleic acid sequence which permits derepression of growth inhibition following expression in the recombinant host cell.

16. Method according to claim 15, characterized in that the recombinant host cell is a plant cell, animal cell, yeast cell, fungal cell or slime fungus cell.

17. Method according to claim 16, characterized in that the animal cell is a mammalian cell or insect cell.

18. Method according to claim 16, characterized in that the yeast cell is a cell of the genus Saccharomyces, Schizosaccharomyces, Kluyveromyces, Hansenula, Pichia, Schwanniomyces, Candida or Yarrowia.

19. Method according to claim 16, characterized in that the fungus cell is a cell of the genus Aspergillus, Neurospora, Rhizopus or Trichoderma.

20. Method according to claim 16, characterized in that the slime fungus cell is a cell of the genus Dictyostelium.

21. Method according to at least one of claims 15 to 20, characterized in that the promoter controlling expression of the gene coding for the marker protein is an inducible promoter.

22. Method according to claim 21, characterized in that the inducible promoter is the PDC1 promoter.

23. Method according to claim 21 and/or 22, characterized in that the recombinant host cells are grown under induced conditions.

24. Method according to at least one of claims 15 to 23, characterized in that the nucleic acid coding for a secretory marker protein is contained in a plasmid with a high copy number.

25. Method according to at least one of claims 15 to 24, characterized in that the DNA fragments employed in step a) of claim 15 are derived from a cDNA gene bank.

26. Method according to claim 25, characterized in that the cDNA gene bank is derived from S. cerevisiae.

27. Method in accordance with at least one of claims 15 to 24, characterized in that the DNA fragments employed in step a) of claim 15 are derived from a genomic gene bank.

28. Method according to at least one of claims 15 to 27, characterized in that the expression vector in step a) of claim 15 containing DNA fragments from any given organism is the CEN/ARS vector.

29. Method according to at least one of claims 15 to 28, characterized in that the secretory marker protein is glucoamylase.

30. Method according to claim 29, characterized in that the nucleic acid sequence coding for the glucoamylase is the GAM1P sequence from S. occidentalis.

31. Use of a recombinant host cell which contains a nucleic acid coding for a secretory marker protein and being under the control of a promoter P2, with the growth of the host cell being inhibited under expression of the gene coding for the marker protein, for identifying nucleic acid sequences which permit derepression of growth inhibition following expression.

32. Host cell, characterized in that it contains a nucleic acid introduced into the cell by a recombinant process, said nucleic acid being selected from the following group:
i) a nucleic acid coding for elF4E from Saccharomyces cerevisiae (CDC33);
ii) a nucleic acid coding for a homologue of elF4E having the biological activity of elF4E;
iii) a nucleic acid derived from the nucleic acid set forth in i) or ii) by degeneration of the genetic code, by deletion, insertion, addition and/or nucleotide exchange, said nucleic acid coding for a polypeptide having the biological activity of elF4E;
iv) a fragment of one of the nucleic acids set forth in i) to iii), said fragment coding for a polypeptide having the biological activity of an elF4E;
v) a nucleic acid capable of hybridizing with a sequence complementary to the nucleic acids set forth in i) to iv);
vi) a nucleic acid coding for CaM kinase from Saccharomyces cerevisiae (CMK2);
vii) a nucleic acid coding for a homologue of a CaM kinase having the biological activity of a CaM kinase;
viii) a nucleic acid derived from the nucleic acid set forth in vi) or vii) by degeneration of the genetic code, by deletion, insertion, addition and/or nucleotide exchange, said nucleic acid coding for a polypeptide having the biological activity of a CaM kinase;
ix) a fragment of one of the nucleic acids set forth in vi) to viii), said fragment coding for a polypeptide having the biological activity of a CaM kinase;
x) a nucleic acid capable of hybridizing with a sequence complementary to the nucleic acids set forth in vi) to ix).

33. Kit, characterized in that it comprises:
(a) an expression vector which comprises a nucleic acid being functionally linked to a promoter and coding for a polypeptide having the biological activity of elF4E, said nucleic acid being selected from the following group:
i) a nucleic acid coding for elF4E from Saccharomyces cerevisiae (CDC33);
ii) a nucleic acid coding for a homologue of elF4E having the biological activity of elF4E;
iii) a nucleic acid derived from the nucleic acid set forth in i) or ii) by degeneration of the genetic code, by deletion, insertion, addition and/or nucleotide exchange, said nucleic acid coding for a polypeptide having the biological activity of elF4E;
iv) a fragment of one of the nucleic acids set forth in i) to iii), said fragment coding for a polypeptide having the biological activity of an elF4E;
v) a nucleic acid capable of hybridizing with a sequence complementary to the nucleic acids set forth in i) to iv). and
(b) a host cell suitable for the secretion of proteins.

34. Kit, characterized in that it comprises:
(a) an expression vector which comprises a nucleic acid being functionally linked to a promoter and coding for a polypeptide having the biological activity of a CaM kinase, said nucleic acid being selected from the following group:
i) a nucleic acid coding for CaM kinase from Saccharomyces cerevisiae (CMK2);
ii) a nucleic acid coding for a homologue of a CaM kinase having the biological activity of a CaM kinase;
iii) a nucleic acid derived from the nucleic acid set forth in i) or ii) by degeneration of the genetic code, by deletion, insertion, addition and/or nucleotide exchange, said nucleic acid coding for a polypeptide having the biological activity of a CaM kinase;
iv) a fragment of one of the nucleic acids set forth in i) to iii), said fragment coding for a polypeptide having the biological activity of a CaM kinase;
v) a nucleic acid capable of hybridizing with a sequence complementary to the nucleic acids set forth in i) to iv). and
(b) a host cell suitable for the secretion and/or glycosylation of proteins.

35. Kit, characterized in that it comprises:
(a) at least one expression vector comprising at least one nucleic acid, said nucleic acid coding for a polypeptide having the biological activity of a CaM kinase and/or elF4E and being selected from the following group:
i) a nucleic acid coding for elF4E from Saccharomyces cerevisiae (CDC33);
ii) a nucleic acid coding for a homologue of elF4E having the biological activity of elF4E;
iii) a nucleic acid derived from the nucleic acid set forth in i) or ii) by degeneration of the genetic code, by deletion, insertion, addition and/or nucleotide exchange, said nucleic acid coding for a polypeptide having the biological activity of elF4E;
iv) a fragment of one of the nucleic acids set forth in i) to iii), said fragment coding for a polypeptide having the biological activity of an elF4E;
v) a nucleic acid capable of hybridizing with a sequence complementary to the nucleic acids set forth in i) to iv);
vi) a nucleic acid coding for CaM kinase from Saccharomyces cerevisiae (CMK2);
vii) a nucleic acid coding for a homologue of a CaM kinase having the biological activity of a CaM kinase;
viii) a nucleic acid derived from the nucleic acid set forth in vi) or vii) by degeneration of the genetic code, by deletion, insertion, addition and/or nucleotide exchange, said nucleic acid coding for a polypeptide having the biological activity of a CaM kinase;
ix) a fragment of one of the nucleic acids set forth in vi) to viii), said fragment coding for a polypeptide having the biological activity of a CaM kinase;
x) a nucleic acid capable of hybridizing with a sequence complementary to the nucleic acids set forth in vi) to ix).
(b) a host cell suitable for the secretion and/or glycosylation of proteins.

36. Kit according to claim 33, characterized in that it further comprises an empty expression vector suitable for the cloning of a nucleic acid coding for a recombinant and/or recombinant secretable protein.

37. Kit, characterized in that it comprises a host cell according to claim 32 and an empty expression vector suitable for the cloning of a nucleic acid coding for a recombinant and/or recombinant secretable protein.
